(19) United States
(12) Patent Application Publication
(10) Pub. No.: US 2022/0275960 A1
(43) Pub. Date: Sep. 1, 2022

(54) AIR QUALITY CONTROL SYSTEM AND METHOD OF OPERATION

(71) Applicants: Ove Arup & Partners Hong Kong Ltd., Kowloon (HK); Henderson Real Estate Agency Limited, Central (HK)

(72) Inventors: Sai Yau Vincent CHENG, North Point (HK); Hiu-Hei Felix CHAN, N.T. (HK)

(21) Appl. No.: 17/673,929

(22) Filed: Feb. 17, 2022

(30) Foreign Application Priority Data
Feb. 26, 2021 (HK) 32021026379.6

(51) Int. Cl. F24F 11/63; F24F 8/22; G01N 33/00; A61L 9/20; H05B 47/105

(52) U.S. Cl. CPC F24F 11/63; F24F 8/22; G01N 33/0027; A61L 9/205; H05B 47/105; F24F 2110/64

(57) ABSTRACT

An air quality control system adapted for use with an entryway for a room, includes a touchless door adapted for association with the room and a door sensor associated with the touchless door to determine whether the touchless door is open or closed. An air purifier selectively deactivates pathogens and pollutants in air flowing through the air purifier and an air quality sensor positioned near the touchless door measures at least one parameter, whereupon the air quality sensor selectively activates and deactivates the air purifier based on whether a threshold level of the at least one parameter is exceeded or not.

### FIELD OF THE INVENTION

The present invention is generally directed to an air quality control system and method of operation. In particular, the present invention is directed to a depuration porch which is provided at a door entryway to maintain high environmental quality inside apartments or other enclosed spaces by using a combination of purification and sensor technology.

### BACKGROUND OF THE INVENTION

In view of the COVID-19 outbreak, the awareness of the importance of air quality is heightened. Contamination from exterior space through entrance to an apartment can occur in one of two ways. The first is by airborne transmission where pollutants, such as particulate matter, volatile organic compounds, and the like; as well as pathogens, such as viruses, bacteria, mold spores, and the like gain access to the apartment by air infiltrating the apartment either when the door is open, such as when a person is entering the apartment, or when the door is closed by infiltration through gaps in the door frame. Regular exposure to fine particulate matter (PM_{2.5} and PM₁₀) provokes inflammation of lung cell membranes, which over long periods of time, leads to higher risk of microbial infections, including COVID-19, as well as increased symptom severity. Additionally, particulate matter (PM) can act as a vehicle for microorganisms, including viruses, such as COVID-19, to be airborne for longer periods of time before being inhaled. The second way for contamination from an exterior space through the entrance is through fomite transmission by surface contact of contaminated occupants or objects. Viruses, bacterial, or mold spores can remain active on surfaces in the range of several hours to several days.

Based on the foregoing, it is clear that there is a need in the art for a system and method to reduce levels of airborne contaminants entering into an indoor space from the outside or exterior. There is also a need to limit the amount of surface contamination which can be imported by occupants or objects carried by the occupants, such as shoes or contaminated hands, as well as to prevent the growth of pathogens on surfaces within the space.

### SUMMARY OF THE INVENTION

One or more embodiments of the present invention provide a depuration porch air quality control system and method of operation.

Yet other embodiments of the present invention provide an air quality control system adapted for use with an entryway for a room, comprising a touchless door adapted for association with the room, a door sensor associated with the touchless door to determine whether the touchless door is open or closed, an air purifier adapted for use in the room, the purifier selectively deactivating pathogens and pollutants in air flowing through the air purifier and in the room, and an air quality sensor positioned near the touchless door, the air quality sensor measuring at least one parameter, wherein the air quality sensor selectively activates and deactivates the air purifier based on whether a threshold level of the at least one parameter is exceeded or not.

A further embodiment of the present invention is to provide a method for purifying air in an entryway, comprising installing a purifier with a power control module in proximity to an entryway door, the power control module receiving input signals from at least an entryway door sensor and an air quality sensor, and determining by the air quality sensor whether a particulate matter (PM) value exceeds at least one predetermined PM value and, if so, turning the air purifier on for a predetermined period of time until the at least one predetermined PM value is no longer exceeded, whereupon the air purifier is turned off.

### BRIEF DESCRIPTION OF THE DRAWINGS

FIG. 1 is a schematic diagram of a depuration porch air quality control system according to the concepts of the present invention;

FIG. 2 is an operational schematic diagram of the depuration porch air quality control system according to the concepts of the present invention; and

FIG. 3 is a flowchart of a method of operation of the depuration porch control system according to the concepts of the present invention.

### DETAILED DESCRIPTION OF THE INVENTION

Referring now to the drawings, it can be generally seen that the present invention is generally directed to a depuration porch air quality control system and method of operation for maintaining a high quality air environment inside an apartment or other enclosed room. Generally, the system includes a photocatalytic oxidizer air purifier which is associated with an appropriate sensor that monitors air quality and a sensor that monitors opening and closing of an entry door. Skilled artisans will appreciate that the present invention may be applicable to any other appropriately sized room, series of rooms or enclosed space.

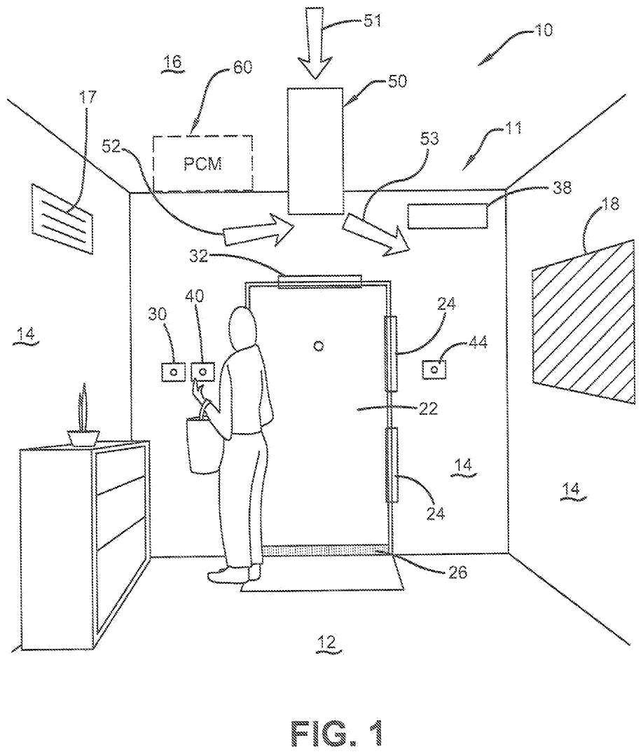

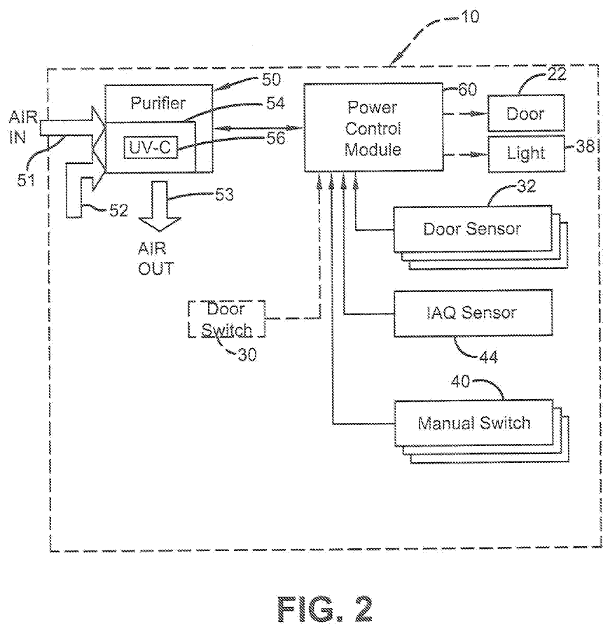

Specifically referring now to FIGS. 1 and 2, it can be seen that an air quality control system used with a room 11 having a porch or other similar type entryway is designated generally by the numeral 10. The room 11 may include a floor 12 with associated walls 14 and a ceiling 16 which further connects the walls 14 to one another. A heating/ventilating air conditioning (HVAC) vent 17 may be disposed in the ceiling, any wall 14, or the floor 12.

In some embodiments, an antimicrobial tile 18 may be associated with one of the surfaces of the room 11. Skilled artisans will appreciate that an entire wall, floor, and/or ceiling surface may be configured as an antimicrobial tile. The tile 18 limits the lifetime and spread of pathogens such as bacteria, viruses, and molds. The antimicrobial tile 18 may be configured with an antimicrobial paint implementing silver ion technology, which includes positively charged silver ions (Ag⁺) into the paint mix. These ions damage the cellular wall of the pathogens on contact and stop cell division by interacting with DNA, rendering the pathogens inactive and inoffensive. Another suitable antimicrobial material used on the tile 18 may be activated bamboo charcoal which features negatively charged ions which help to trap the pathogens inside its porous structure. The activated bamboo charcoal may be incorporated into the paint mix and can also be used as a suitable alternative. Other floor or wall surfaces may be vinyl or ceramic and may also feature antimicrobial paint as part of their material fabrication, offering antimicrobial properties for floors as well. Both Ag⁺ and activated bamboo charcoal are believed to be harmless for occupant health. Other antimicrobial agents in the art may be applied onto the antimicrobial tile as skilled artisans will appreciate.

Associated with the room 11 may be a door designated generally by the numeral 22. Such a door may be provided with features to limit the number of contact points on the depuration porch, as well as to reduce the ingress of contaminated air. Such features may include a soft hinge 24 which eliminates the need for closing the door by hand. The door 22 may also be provided with a door seal 26 which is placed at the top and/or bottom of the door to prevent contaminated air from entering the room while the door is closed. The door 22 may include a door lock/switch 30 which may be in the form of a touchless infrared (IR) switch which is placed near the entrance to provide manual control of the door's operation. A similar switch may be provided at the room's exterior. It will further be appreciated that opening and closing of the door 22 may be controlled by a key card, a phone application or other means of control input in the art.

A door opening sensor 32 is associated with the door 22 to automatically control lighting and air purification and to limit contact points. Upon the door's opening, the sensor 32 will detect this and automatically turn on lighting for a duration of 10 minutes or other predetermined period, or until manually switched off. Actuation of the door opening sensor may also be utilized to control operation of a purifier as will be discussed. A light 38, which may be separately powered, is provided in the room and may be further controlled by a touchless light switch 40. The switch 40 may also control the operation of the purifier allowing the occupant to switch on and off of the purifier manually as desired. It will further be appreciated that operation of the light 38 and purifier 50 may be controlled by a remote control, phone application or other means of control input in the art.

An indoor air quality (IAQ) sensor is designated generally by the numeral 44 and may be situated proximate to the doorway or anywhere within the room 10. The sensor 44 measures levels of particulate matter such as PM_{2.5} and PM₁₀, or any other indoor air quality parameters such as ozone. As used herein, PM_{2.5} refers to fine inhalable particles that are generally 2.5 micrometers and smaller; and PM₁₀ refers to inhalable particles with diameters that are generally 10 micrometers and smaller. The sensor controls operation of the purifier through a module as will be discussed.

An air purifier 50 may be placed in proximity to the doorway and in some embodiments inside a suspended ceiling with a vent grate below. The purifier may be a photocatalytic oxidizer (PCO) air purifier or other air purifier as will appreciated by skilled artisans. The purifier 50 may have an external air source inlet 51 and/or may have an internal air source inlet 52. The purifier 50 also has an exhaust air outlet 53 which exhausts purified air into the room 11 in close proximity to the door and its opening.

As best seen in FIG. 2, the PCO purifier 50 is controlled by a power control module (PCM) 60 which sets the purifier's ON/OFF status based on signals from at least the door sensor 32, the manual switch 40, and/or the IAQ sensor 44. The power control module 60 and associated components provide the necessary hardware, software, and memory to implement operation of the air quality control system 10. The purifier 50 comprises a fan to draw air into a photocatalytic chamber 54 which contains a UV-C lamp 56 and a photocatalyst material. In one embodiment, the photocatalyst material is titanium dioxide (TiO₂). One skilled would appreciate other photocatalyst materials may be used in the present invention. Not only does the UV-C lamp 56 decompose incoming pathogens by degrading their DNA, the reaction of the air with the photocatalytic material in the chamber 54 under the influence of the UV-C lamp also produces charged ions which are emitted downward through the exhaust vent 53, forming a “curtain” across the doorway. The charges ions may include but are not limited to OH⁻ and O₂⁻. Other charges ions may be produced depending on the types of purifier is installed. These charged species deactivate airborne pathogens (viruses, bacteria, and mold spores) by degrading their cellular membrane. In the case of viruses such as COVID-19, the protein coat of individual virus is degraded to disable their infectious potential (and produces water and CO₂ as by-products). The charged species also cluster around other organic or inorganic particulate matter, causing them to accumulate mass and harmlessly fall to the ground where they can be removed periodically by regular cleaning. The charged species may also be dispersed into the room and react with airborne and fomite-borne contaminants. The lifetime of these species is shorter in highly contaminated air as they react upon contact with pathogens and other particulates, but in clean air, can typically remain airborne and active in space for over one hundred seconds. In view of this lifetime, the charged species can also react with pathogens on surfaces and fomites, such as clothes and furniture, in the room.

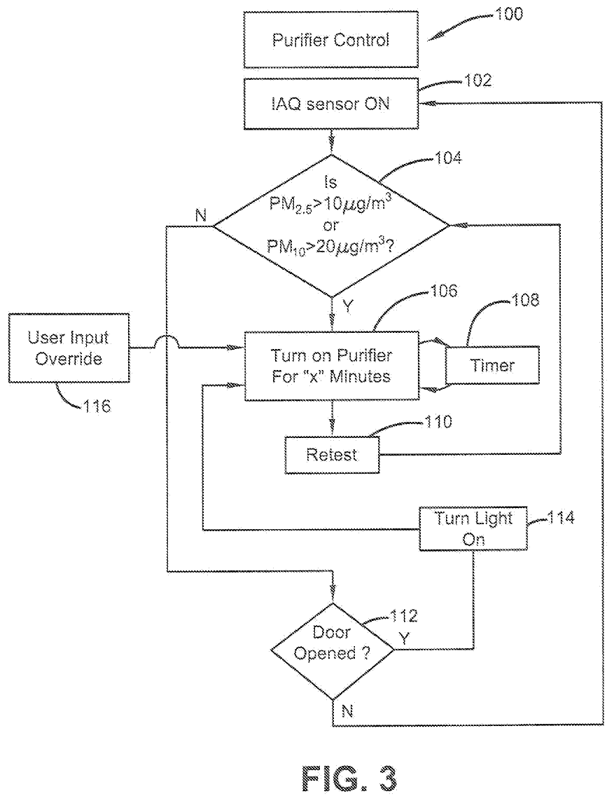

Referring now to FIG. 3, it can be seen that a method of operation for the air quality control system is designated generally by the numeral 100. Initially, the IAQ sensor 44 is turned on at step 102 and it periodically samples the air within the apartment to determine whether certain particulate levels have reached a predetermined threshold value. In one embodiment, the sensor 44 tests whether the PM_{2.5} value is greater than 10 μg/m³ and/or whether the PM₁₀ value is greater than 20 μg/m³. If either value is exceeded, then the purifier 50 is turned on at step 106 for a predetermined amount of time. The predetermined amount of time may be a time frame needed for the enclosed space to restore to a safe PM threshold level or a time frame preset of the user. In one embodiment, the purifier is set to be turned on for ten to thirty minutes which is the tested time frame needed for an enclosed space of approximately 200 ft²-500 ft² to restore to a safe PM threshold level. Other predetermined amount of time may be set based on the size of the enclosed space as will appreciated by a skilled artisan. This time period which may be set by the user with timer 108 and the purifier operates until the timer expires. Skilled artisans will appreciate that other threshold values or other indoor air quality parameters may be set to control the operation of the purifier. Upon expiration of the time, then at step 110, a retest is initiated wherein the purifier 50 is turned off and the process returns to step 104 to determine whether the values have been reduced below the desired thresholds. Once the tested values are below the threshold values, then the process continues to step 112 to determine whether the door has been opened or not. After values are tested to be below the threshold values or during sampling by the IAQ sensor, if the door has been opened, then presumably new ambient air has entered the enclosed space and the process goes to step 114 where at least one of the lights 38 is automatically turned on. Next, the process returns to step 106 and the purifier 50 is turned on for the predetermined period of time. During this time, or any other time, the user at step 116 may override operation of the purifier 50 and/or the light(s) 38 and turn them on or off manually by touchless switch 40 or any other type of switch in the art. In any event, upon completion of the time, then the process returns to step 104 through the retest step 110 to determine whether the threshold values are exceeded or not. Once the threshold values are determined to not exceed the predetermined values and the door is not opened as determined at step 112, the process returns to step 102 so as to continue monitoring the air quality within the room. The IAQ sensor may monitor other indoor air quality parameters, such as ozone, and follow similar operations as described above.

Based on the foregoing, the advantages of the present invention are readily apparent. The configuration disclosed herein reduces the high risk potential for contaminants as defined above entered through doorway and eliminates them as soon as they enter a room. Such configurations do not produce ozone and, as such, do not impact the human inhabitants. The system provides for minimal maintenance requirements and is further enhanced by the ability to control and interlock occupancy habit. The system also allows for controlling and monitoring with a sensor.

Various modifications and alterations that do not depart from the scope and spirit of this invention will become apparent to those skilled in the art. This invention is not to be duly limited to the illustrative embodiments set forth herein.

What is claimed is:

1. An air quality control system adapted for use with an entryway for a room, comprising:
a touchless door adapted for association with the room;
a door sensor associated with said touchless door to determine whether said touchless door is open or closed;
an air purifier adapted for use in the room, said purifier selectively deactivating pathogens and pollutants in air flowing through said air purifier; and
an air quality sensor positioned near said touchless door, said air quality sensor measuring at least one parameter, wherein said air quality sensor selectively activates and deactivates said air purifier based on whether a threshold level of said at least one parameter is exceeded or not.

2. The system according to claim 1, further comprising:
a power control module which receives input from said door sensor and said air quality sensor and generates output to control operation of said air purifier.

3. The system according to claim 2, further comprising:
a touchless switch adapted for association with the room and generating input for receipt by said power control module to control operation of said air purifier.

4. The system according to claim 3, wherein said air quality sensor detects at least one particulate matter level and turns said air purifier on for a predetermined period of time when said at least one particulate matter level exceeds a predetermined particulate matter threshold.

5. The system according to claim 3, wherein said door sensor detects opening of the touchless door and turns said air purifier on for a predetermined period of time when said touchless door is opened.

6. The system according to claim 3, further comprising:
a light adapted for use in the room, wherein operation of said light is controlled by said power control module and wherein said power control module receives an input from said door sensor when said door sensor detects opening of the touchless door and turns said light on for a predetermined period of time.

7. The system according to claim 6, wherein said touchless switch generates input for receipt by said power control module to control operation of said light.

8. The system according to claim 1, further comprising:
an antimicrobial tile adapted for association with the room.

9. The system according to claim 1, wherein said air purifier has an outlet to exhaust purified air into the room.

10. The system according to claim 9, wherein said air purifier has an inlet to draw air in from the room.

11. The system according to claim 7, wherein actuation of said touchless switch terminates said predetermined period of time and turns off one of said air purifier or said light or both or actuation of said touchless switch turns on one or said air purifier or said light or both.

12. The system according to claim 1, wherein said air purifier comprises a photocatalytic chamber containing a UV-C lamp through which the air flows.

13. A method for purifying air in an entryway, comprising:
installing a purifier with a power control module in proximity to an entryway door, said power control module receiving input signals from at least an entryway door sensor and an air quality sensor; and
determining by said air quality sensor whether a particulate matter (PM) value exceeds at least one predetermined PM value and, if so, turning said air purifier on for a predetermined period of time until said at least one predetermined PM value is no longer exceeded, whereupon said air purifier is turned off.

14. The method according to claim 13, further comprising:
detecting opening of said entryway door by said entryway door sensor; and
turning said air purifier on for a predetermined period of time.

15. The method according to claim 14, further comprising:
turning said purifier off by a user prior to expiration of said predetermined period of time or turning said purifier on by a user.

16. The method according to claim 14, further comprising:
providing at least one seal between said entryway door and a floor of the entryway.

17. The method according to claim 14, further comprising:
positioning an antimicrobial tile in said entryway.

18. The method according to claim 14, further comprising:
flowing the air in the purifier through a photocatalytic chamber containing a UV-C lamp.
